(19) United States
(12) Patent Application Publication
(10) Pub. No.: US 2026/0131484 A1
(43) Pub. Date: May 14, 2026

(54) ROBOT BASE AND MEDICAL OPERATING SYSTEM

(71) Applicant: KARL STORZ SE & Co. KG, Tuttlingen (DE)

(72) Inventor: Martin HOFFMANN, Berlin (DE)

(73) Assignee: KARL STORZ SE & Co. KG, Tuttlingen (DE)

(21) Appl. No.: 19/384,649

(22) Filed: Nov. 10, 2025

(30) Foreign Application Priority Data
Nov. 11, 2024 (DE) 10 2024 132 852.5

(51) Int. Cl. B25J 15/00; A61B 34/00; B25J 9/00

(52) U.S. Cl. CPC B25J 15/0052; A61B 34/70; B25J 9/0084

(57) ABSTRACT

The present invention relates to a robot base comprising a first robot arm interface which is designed to couple a first robot arm and at least a second robot arm interface which is designed to couple a second robot arm, wherein the first robot arm interface and the second robot arm interface are each designed to couple a software RCM robot arm. The present invention further relates to a robot base comprising a holding arm on which the first robot arm interface and the second robot arm interface are formed in a fixed position relative to one another.

### CROSS-REFERENCE TO RELATED APPLICATIONS

This application claims priority of German Patent Application No. DE 102024132852.5 filed on November 11, 2024, the contents of which are incorporated herein.

### TECHNICAL FIELD

The present disclosure relates to a robot base and to a medical operating system.

### BACKGROUND

Robot bases for medical operating systems, which support a plurality of robot arms, are known in the prior art. Each robot arm has a medical instrument such as an endoscope, a surgical tool, or the like attached to the distal end, which allows for minimally invasive surgical procedures.

To perform the minimally invasive procedure using such a medical operating system, the patient is fixed to an operating table. The operating system is positioned next to the operating table, and the robot arms are individually aligned and adjusted for the patient.

Such medical operating systems from the prior art typically have a so-called "Hardware Remote Center of Motion" (hardware RCM). A hardware RCM describes a virtual pivot point about which a robot arm or a medical instrument fastened to the robot arm moves. This pivot point remains stationary during the movement. The hardware RCM is achieved using a specially designed mechanical structure and is based, for example, upon the kinematics of a parallelogram. Parallel kinematic robots are described, for example, in US 5697939 A, US 5817084 A, US 6902560 B1, and US 2016/0100900 A1.

A medical instrument fastened to the robot and inserted into the patient's body to perform the procedure is moved about this hardware RCM and, in a preparatory procedure, adjusted so that it lies at a point where the medical instrument enters the body. This ensures precise control of the medical instrument fastened to each robot arm, minimizing forces exerted on the patient's skin or tissue.

### SUMMARY

The present disclosure is based upon the knowledge that these conventional medical operating systems are bulky and heavy due to the mechanics required for providing the hardware RCM. This sometimes makes it impossible to use known medical operating systems in relatively small operating theaters and also requires a high minimum load-bearing capacity of a floor on which the system is to be placed. Furthermore, the size of the system makes it difficult for a doctor to access the patient, even in larger operating theaters. This is particularly unfavorable for anesthesia. Furthermore, the inventors recognized that, when converting to an open procedure, which may be necessary due to a complication, for example, too much time is required to remove a conventional medical operating system from the patient due to the size of the system.

Proceeding from the prior art, the object addressed by the disclosure is to provide a compact medical operating system.

The object is achieved according to the disclosure by a robot base and a medical operating system as are described herein and defined in the claims.

The present disclosure provides for the provision of a robot base, in particular for a medical operating system. Said base comprises a first robot arm interface, which is designed to couple a first robot arm, and at least a second robot arm interface, which is designed to couple a second robot arm.

According to one aspect of the disclosure, the first robot arm interface and the second robot arm interface are each designed to couple a software RCM robot arm.

According to a further aspect of the disclosure, the robot base comprises, in particular in addition to the features of the aspect described above, a holding arm on which the first robot arm interface and the second robot arm interface are formed in a fixed position relative to one another.

The present disclosure further provides for the provision of a medical operating system. Said system comprises a robot base according to the disclosure and a first software RCM robot arm, which is coupled to the first robot arm interface.

These features allow for a compact medical operating system. The robot base according to the disclosure has small dimensions and takes up little space, especially compared to conventional robot bases for hardware RCM robot arms. This can save upon weight, both in absolute terms and relative to a base surface of a mounting unit, by means of which the robot base is placed on the floor of an operating theater. The robot base according to the disclosure and the corresponding medical operating system that uses this robot base can therefore be used in a wider range of operating theaters. In other words, the use of the robot base and operating system places less of a demand upon the operating theater in terms of its size and the floor load-bearing capacity compared to conventional operating systems. Furthermore, the use of software RCM robot arms has the advantage that the operating system offers great flexibility in terms of the movement of the robot arms and their adjustment to the patient. This, combined with the largely compact design of the robot base and the operating system, means that a doctor can reach the patient more quickly in an emergency - for example, to initiate life-saving measures. Furthermore, greater flexibility can be provided with regard to the placement of the robot base in the operating theater, and the operating system can be set up quickly.

The medical operating system can, in particular, be a robot-assisted one, for example, for performing minimally invasive procedures or for minimally invasive surgery. Such a procedure is, in particular, an operative, surgical procedure that is performed within a body cavity of a patient. One example of such a procedure is the gastric bypass. As already described, these procedures involve introducing a number of medical instruments into the body cavity through only a small incision. The medical instruments are arranged and/or formed on robot arms. The medical instruments can be positioned and controlled within the body cavity via a control system for the robot arms.

A robot base can be a central platform for a medical, in particular robot-assisted, operating system. For example, it provides the necessary stability to support the robot arms, can be designed to isolate or decouple vibrations from the floor, and can enable tilt-proof placement and adjustment of the robot arms. This enables precise, accurate movements of the robot arms during surgical procedures. In other words, the robot base can be regarded as a mechanical mounting unit.

For example, if the robot arms of the operating system are not positioned above the base surface of the robot base, the robot base can form a counterweight to the robot arms. This is particularly important with regard to the weight of the robot arms. If the robot arms are designed to be lightweight, a small, lightweight robot base can be provided. In this case, software RCM robot arms are used that can be compact and lightweight. This ensures that the robot base, for one, needs to provide a relatively low counterweight to the robot arms and, for another, requires a relatively small base surface.

According to some embodiments, the robot unit and/or the medical operating system can be movable in an operating theater or similar premises, or can be movable within an operating environment and positionable relative to a patient. Typically, a patient is fixed to an operating table, or the patient is on the operating table during a procedure. The robot base can, in particular together with robot arms attached thereto, be positioned relative to this operating table - for example, after the patient has been prepared for the procedure. In this context, the compact operating system and the compact robot base result in a great degree of flexibility and consequently less time when preparing for the procedure. Furthermore, the compact design provides open access to the patient during the procedure.

The robot base can house additional functional units, such as a control unit, one or more power supply units, an energy store, and/or the like. The robot arms or medical instruments fixed to the robot arms can be operable by means of the control unit and/or the at least one power supply unit.

A robot arm can be understood to mean a movable device that can be controlled through several axes and can perform precise movements in different directions. It may be designed to support and position one or more medical instruments such as a surgical tool, e.g., forceps, an RF tool, a laser head, and/or the like, as well as an endoscope. These instruments can be inserted into a body cavity of a patient to perform a minimally invasive procedure. Typically, a plurality of medical instruments are used for a minimally invasive procedure. These are often provided on different robot arms. The robot arms can be movable in a coordinated manner adapted to match one another and collectively controlled during the procedure. Each instrument can be inserted, for example, into the body cavity via an individual or even common access point, which may, for example, be an incision.

For example, if a procedure is performed in the abdominal cavity, a doctor can make a small, targeted incision in the abdominal wall through which an instrument can be introduced into the abdominal cavity. The instrument is usually rotated about this access point in the abdominal wall to prevent stress and injury to the abdominal wall tissue. This access point can be understood as a so-called trocar point. The medical instrument can therefore be moved in a cone-shaped region within the abdominal cavity, with the trocar point describing the apex of the cone.

A robot arm can be accordingly designed to be moved about the trocar point. This can be located at a distance from the robot arm in the space. The trocar point can be individually adjusted for each robot arm - for example, during preparation for the operation. As already described, it is important to rotate the robot arm about this trocar point and to perform linear movements through the trocar point. Otherwise, there is a risk of injury to the patient.

As previously described, a hardware RCM robot arm can be moved about the RCM. In accordance with the above statements, the RCM can map the trocar point, and the robot arm or a medical instrument arranged on the robot arm can be rotated about the RCM.

A software RCM robot arm can also be movable about the trocar point. Unlike the hardware RCM robot arm, this cannot be achieved through the special mechanical design, but, rather, through software-based control of the robot arm, for example. This means that the fixed pivot point of the medical instrument, by which movements are to be performed precisely about a specific point (e.g., the trocar point), is determined and maintained, for example, by control algorithms and software control.

Instead of using mechanical devices such as parallel kinematics, the software used instead can calculate the required robot arms’ movements and continuously adjust them to ensure that the selected RCM or trocar point remains stable and is maintained.

A robot arm interface can be provided for mechanically fixing or fastening the robot arm to the robot base. Additionally, the robot arm can be electrically connectable to the robot base via the robot arm interface. The robot arm interface can therefore be used, for example, to transmit electrical energy and/or control signals. Accordingly, the robot arm can be electrically connectable to a robot arm control unit via the robot arm interface.

The robot arm interface can, for example, form a connecting unit together with a holding portion of the robot arm. The holding portion can define a proximal portion of the robot arm. A medical instrument may be arranged on a distal portion.

For example, the connecting unit can be designed as a type of bayonet connection. Alternatively or additionally, the connecting unit can have a clamping mechanism and/or a screw mechanism. Other methods of fixing the robot arm to the robot base via the robot arm interface are also conceivable.

The term "holding arm" can be understood to mean a carrier unit of the robot base for a plurality of robot arms. The holding arm can accordingly be structured such that the robot arms can be held securely or rigidly with respect to one another, in particular with regard to the holding portions of the robot arms. In particular, the term "holding arm" is understood to mean a beam-shaped structure, or the holding arm can be beam-shaped. The holding arm can therefore be an elongated structure that has a greater extent along one axis than along the axes perpendicular to said axis, for example. Said axis can therefore be a main axis of extension. Along this axis, the extension of the holding arm can, for example, be at least twice, in particular at least three times, preferably at least four times, as large as along an axis perpendicular to this main axis of extension.

The holding arm can be alignable in such a way that it extends away from the base surface of the robot base. The holding arm allows the robot arm interfaces to be positioned in a space above a surface that is at a distance from the base surface. For this purpose, the holding arm can be connected to other components of the robot base, in particular at one end. The opposite end of the holding arm can be freely movable in the space. The "end" and the "opposite end" refer in particular to the ends in the longitudinal direction of the holding arm.

The position of the first robot arm interface relative to the second robot arm interface can be fixed. Advantageously, the two robot arm interfaces can therefore be collectively aligned in the space. This makes it easier to adjust the medical operating system. The term "position" can refer to a position in the space and/or an angular orientation. In other designs, the robot arms may not be movable relative to one another and/or each be immovably arranged in an intended position on the robot base. If the holding arm is provided, the first robot arm interface and the second robot arm interface are formed on the holding arm in a fixed position relative to one another, as per the feature mentioned above. By positioning the holding arm relative to other components of the robot base, in particular a main body of the robot base, the robot arm interfaces can therefore be positioned in the space.

A compact robot base that is flexible to use can be provided if the first robot arm interface and the second robot arm interface are arranged side-by-side along a longitudinal axis of the holding arm. In other words, the robot arm interfaces can be provided along the longitudinal direction of extension or along the main axis of extension of the holding arm.

The robot base can also comprise a third robot arm interface, which is designed to couple a third robot arm. The third robot arm interface can, in particular according to the above statements, be formed on the holding arm in a fixed position relative to the first and second robot arm interfaces. Therefore, another robot arm can be provided on the holding arm. This allows for an increase in the number of medical instruments, thereby enabling more complex procedures. According to some embodiments, the robot base can have exactly three robot arm interfaces. These can be formed on the holding arm and can accordingly be collectively movable in the space by positioning the holding arm.

According to a development, the first robot arm interface, the second robot arm interface, and the third robot arm interface are arranged side-by-side on the holding arm so as to be equally spaced apart from one another along the longitudinal axis. This allows for an even distribution of the forces acting upon the holding arm and optimized use of space. This makes it possible to optimize the weight of the holding arm, resulting in a more compact robot base. Furthermore, the equidistant arrangement leads to improved stability of the system and facilitates collision avoidance between the robot arms, thus enabling precise and unimpeded movements during a surgical procedure.

A compact operating system and a robot base that can be moved efficiently in the space can be provided if the holding arm can be arranged suspended above an operating table, in particular in such a way that the robot arm interfaces can be arranged in an imaginary space extending vertically above the operating table and laterally delimited by the operating table. In a stowed configuration, the medical operating system can be arranged in a space-optimized manner, for example. For example, the robot arms can be stored close to the robot base, and/or the holding arm can be stored in a space-saving manner. In one application configuration, the holding arm can extend away from the main body of the robot base. If necessary, the holding arm can therefore be movable across the operating table to transfer the operating system into the application configuration. The term "suspended" can be understood to mean that the holding arm is arranged to extend away from the main body of the robot base. In other words, the holding arm is arranged in a state where it extends laterally away from the main body. If the main body is arranged with its base surface next to the operating table, the holding arm can accordingly extend approximately across the operating table and across a patient lying on the operating table, in particular in the application configuration.

Furthermore, the robot base can comprise a mounting unit for placement on a floor. The holding arm can be connected to the mounting unit and have at least three, in particular exactly three, degrees of freedom with respect to the mounting unit. The ability to position the holding arm with three degrees of freedom allows the robot arms to be flexibly oriented with respect to the patient and the operating table. The ability to move the holding arm makes it possible to use a compact robot base that is nevertheless flexible and allows for a wide range of procedures. The main body of the robot base can comprise and/or define the mounting unit. The mounting unit can also have and/or define the base surface.

Height adjustability of the holding arm can be achieved if the holding arm is movable in a vertical direction relative to the mounting unit, in particular along a vertical axis which extends through the mounting unit. This allows the height of the robot arm interfaces to be adjusted relative to the patient. This opens up the possibility, for example, of flexibly adapting the operating system to local conditions and aligning the robot arms with regard to the patient's lying position. The patient can therefore lie horizontally or at an incline, wherein the height adjustability also makes it possible to perform a procedure in the head region as well as in the leg region when the patient is in an inclined position. The holding arm can be moved in a vertical position in a range of, for example, 500 nm to 2,500 nm, in particular 700 mm to 2,000 mm, preferably 900 nm to 1,500 mm. The term "vertical position" can refer in particular to the distance between a point where the holding arm couples to other components of the robot base and the floor on which the robot base is placed. Because the vertical axis extends through the mounting unit, good statics and stability of the operating system can be achieved. For example, the robot base can comprise an extendable column to which the holding arm is movably fastened. The column can be extended vertically out of the mounting unit in a telescopic manner. The column may have a structure at its distal portion used to hold and movably mount the holding arm.

According to some embodiments, the holding arm is arranged on the mounting unit in such a way that the longitudinal direction of the holding arm extends orthogonally to the vertical axis. In other words, the main axis of the holding arm extends perpendicularly to the vertical direction, or the holding arm is arranged horizontally. The robot arm interfaces can therefore be arranged side-by-side in the horizontal plane, in particular equidistantly from one another.

According to a development, the holding arm can be positioned in a horizontal plane by the holding arm being rotatable relative to the mounting unit about a vertical axis, which extends in particular through the mounting unit. In other words, the holding arm can be pivoted about the vertical axis. The holding arm can be rotated about the vertical axis by 360 degrees. This means that the holding arm can assume any angular position in the horizontal plane. This is especially true if the holding arm extends in the horizontal plane, in particular by means of the main axis, or if the longitudinal axis extends in the horizontal plane.

Furthermore, the holding arm can be rotatable about its longitudinal axis, wherein the longitudinal axis is in particular perpendicular to a vertical axis which extends through the mounting unit. This allows the robot base to be used in a flexible manner. For example, the robot base could be placed either to the left or right of an operating table. Accordingly, the holding arm, whose longitudinal axis extends horizontally, can be pivoted across the operating table. The vertical position can be adjusted accordingly. In addition, the holding arm can be rotated about its longitudinal axis to bring the robot arm interfaces into the desired position relative to the operating table. Accordingly, for the position to the left and right of the operating table, the holding arm can be brought into a similar orientation with respect to the operating table. The robot arm interfaces can therefore, for example, point toward the same end of the operating table in both orientations.

Such a robot base is therefore characterized by a high degree of flexibility. Its use places minimal demands upon the operating environment. In particular, the space required can be reduced.

The holding arm can be movable by motor in the three degrees of freedom mentioned. Therefore, motors can be provided that can be specifically actuated to orient the holding arm. Furthermore, the holding arm can be holdable in a specific orientation. For example, the robot base can comprise a locking mechanism in particular for each degree of freedom, by means of which the holding arm can be fixed.

Complex procedures can be made possible and a high degree of flexibility of the operating system can be achieved if the software RCM robot arm has at least seven degrees of freedom. This allows the robot arm to be moved about the trocar point or the RCM with a large degree of freedom of movement. By coordinating the actuation of individual joints or segments of the robot arm, several movement patterns can be executed, all of which allow adherence to the RCM. This can reduce the space required, as it allows for flexible and quick responses to external influences. For example, the robot arm can be operated close to a wall. Similarly, a collision with a doctor can be prevented by having the robot arm avoid the doctor while still adhering to the RCM. In other words, the robot arm can have a large null space due to the at least seven degrees of freedom. In some embodiments, the software RCM robot arm has exactly seven degrees of freedom.

The null space of a robot arm can refer to the set of movements that the robot arm can perform without changing the position or orientation of an end effector. This means that the RCM can be maintained during different movements. A suitable robot arm, in particular one with multiple joints, can have more degrees of freedom than necessary, to achieve a specific position and orientation of the end effector. The null space contains movements that are performed within these additional degrees of freedom, while the end effector remains stable.

For example, in a robot arm with seven degrees of freedom, a specific position of the end effector can be achieved in different ways or by different movements by aligning the joints differently. These different joint positions, which leave the end effector unchanged, belong to the null space. Such movements in the null space can be used to avoid collisions, minimize joint stress, or to optimally hold the robot arm.

Six of the degrees of freedom can be degrees of rotational freedom. This allows for a large null space while at the same time achieving a compact robot arm design. The software RCM robot arm can therefore have six joints, by means of which an angular position of two adjacent segments of the robot arm can be set. Three of the degrees of rotational freedom can allow adjacent segments to rotate about a longitudinal axis of the segments. Three of the degrees of rotational freedom can again allow adjacent segments to pivot perpendicularly to the longitudinal axis of the segments. The robot arm can, for example, be formed of seven segments, with one of the segments forming the holding portion. An additional degree of freedom can enable linear movement. The linear movement can be achieved, for example, by means of a distal end segment of the robot arm. This segment may have a rail along which a medical instrument fastened thereto can move linearly. The medical instrument may also have one degree of rotational freedom about its longitudinal axis.

The medical operating system can have a second software RCM robot arm, which has a corresponding design to the first software RCM robot arm and is coupled to the second robot arm interface. Accordingly, the medical operating system can have a third software RCM robot arm. Because the robot arms have a large null space, they can be flexibly moved relative to one another, thus preventing a collision between the robot arms without significantly limiting the possible applications of the robot arms. This enables the safe operation of the operating system, allowing for a wide variety of procedures.

The present disclosure is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects. Accordingly, for example, a first object and a third object may be included, but not a second object. However, a number and/or sequence of objects could also be derived using numerical words.

### BRIEF DESCRIPTION OF DRAWINGS

FIG. 1 is a schematic perspectival view of a medical operating system comprising a robot base and three software RCM robot arms as well as an operating table;

FIG. 2 is a schematic perspectival view of the robot base;

FIG. 3 is a schematic bottom view of the medical operating system having the robot base and the operating table;

FIG. 4 is a schematic view of one of the software RCM robot arms and a medical instrument arranged at the distal end thereof;

FIG. 5 is a schematic frontal view of the medical operating system and the operating table; and

FIG. 6 is a schematic lateral view of the medical operating system and the operating table.

### DETAILED DESCRIPTION OF PREFERRED EMBODIMENTS

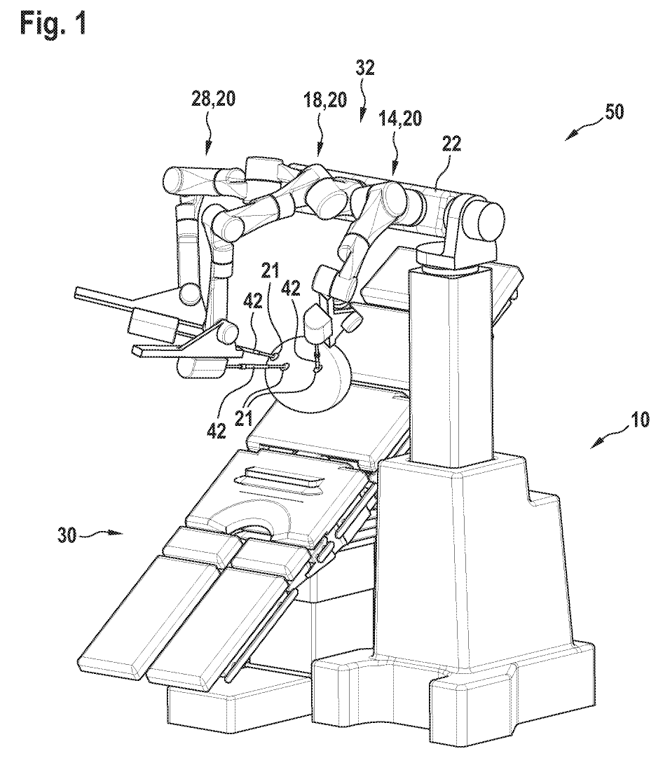

FIG. 1 is a schematic perspectival view of a medical operating system 50 comprising three software RCM robot arms 20 and a robot base 10. The three software RCM robot arms 20 are coupled to the robot base 10 - more precisely, to a holding arm 22 of the robot base 10. Furthermore, an operating table 30 can be seen, on which a patient (not shown) lies when undergoing a procedure. In the case shown, the operating table 30 is at an angle to the horizontal.

Using the medical operating system 50, a minimally invasive procedure can be performed on the patient - for example, a gastric bypass in the configuration of the medical operating system 50 shown. For this purpose, a medical instrument 42 is arranged at the distal end of each of the software RCM robot arms 20. This can be moved about a corresponding trocar point 21 by means of the associated software RCM robot arm 20. The trocar point 21 provides access to a body cavity or the abdominal cavity of the patient, within which the procedure is to be performed. During the procedure, the medical instrument 42 is not intended to move laterally relative to the associated trocar point 21, to avoid injury to the patient's tissue. Accordingly, an RCM of the associated software RCM robot arm 20 can be placed on the trocar point with the assistance of software so that, when it moves, it moves the medical instrument 42 about the trocar point 21 and accordingly the RCM. Within the body cavity, movement of the medical instrument can thereby cover a cone-shaped region, the apex of which lies at the trocar point.

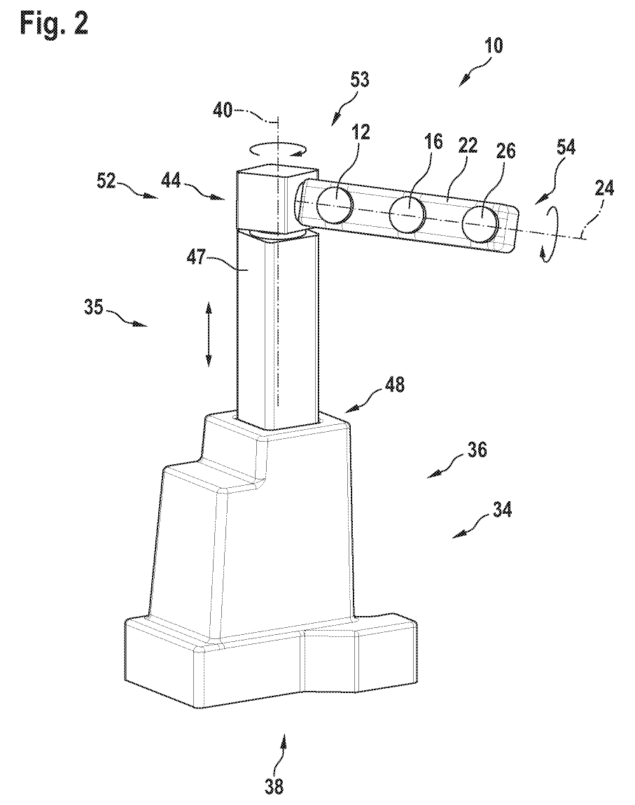

The holding arm 22 is arranged suspended above the operating table 30. That is, it extends above the operating table 30 and transversely thereto. This means that the software RCM robot arm 20 is coupled to the holding arm 22 above the operating table 30. The holding arm 22 comprises three robot arm interfaces (see, for example, FIG. 2) to which the software RCM robot arms 20 are coupled. These robot arm interfaces are therefore arranged in an imaginary space 32 extending vertically above the operating table 30 and laterally delimited by the operating table 30.

The software RCM robot arms 20 each have seven degrees of freedom, as will be described in more detail below. As a result, they have a large null space. This provides several different movements during the procedure, while maintaining the trocar point 21 or the RCM. This can prevent collisions between the software RCM robot arms 20, while still maintaining a high degree of flexibility.

Before a procedure, the patient can be placed on the operating table 30 and prepared for the procedure. Only after the patient has been prepared must the medical operating system 50 be placed next to the operating table 30. Since the robot base 10 is designed to couple the software RCM robot arms 20, the operating system 50 and the robot base 10 are compact. This means the operating system 50 can be positioned easily and efficiently. This can therefore save time when preparing for the procedure. As will be described in more detail below, the holding arm 22 has three degrees of freedom, or can be flexibly positioned in the space. This allows the operating system 50 to be flexibly placed next to the operating table 30. For example, the robot base 10 can be placed to the left or right of the operating table 30 without restricting the functionality. The robot arm interfaces can then be specifically arranged for the position of the robot base 10 relative to the operating table 30 by aligning the holding arm 22.

FIG. 2 is a schematic perspectival view of the robot base 10. The robot base 10 has a mounting unit 34 for placement on a floor. The mounting unit 34 forms a main body 36 of the robot base 10. Furthermore, it has on its underside a base surface 38, which is in contact with the floor.

A linear guide 48 is formed in the main body 36 and extends along a vertical axis 40. The vertical axis 40 extends through the robot base 10. The linear guide 48 serves to linearly guide a movable column 47 of the robot base 10. The column 47 can be driven by motor to move linearly along the vertical axis 40. For this purpose, the robot base 10 has a motor (not shown).

The robot base 10 also has the holding arm 22, on which robot arm interfaces 12, 16, 26 are arranged. Each of the robot arm interfaces 12, 16, 26 can be coupled to a software RCM robot arm 20. A first robot arm 14 can be coupled to a first robot arm interface 12, a second robot arm 18 can be coupled to a second robot arm interface 16, and a third robot arm 28 can be coupled to a third robot arm interface 26 (see also FIG. 1). The holding arm 22 is beam-shaped and extends along its longitudinal axis 24. This forms a main axis of the holding arm 22. The robot arm interfaces 12, 16, 26 are arranged side-by-side along the longitudinal axis 24 so as to be at equal distances from one another. The centers of the robot arm interfaces 12, 16, 26 are spaced approximately 300 mm apart, and the beam-shaped holding arm 22 is approximately 700 mm long. The position of the robot arm interfaces 12, 16, 26 relative to one another is therefore fixed.

The column 47 is part of a positioning unit 35, by means of which the holding arm 22 can be aligned. The positioning unit 35 or the robot base 10 has the holding arm 22. This is fastened by its proximal end 53 to a distal portion 52 of the column 47 by means of two pivot joints 44, 46 and can move relative to the column 47 with two degrees of freedom. The first pivot joint 44 allows the holding arm 22 to be rotated about the vertical axis 40. The second pivot joint 46 allows the holding arm 22 to be rotated about its longitudinal axis 24. The holding arm 22 is therefore held at its proximal end 53. Its distal end 54 is freely suspended in the space. The longitudinal axis 24 of the holding arm 22 is perpendicular to the vertical axis 40 and extends in a horizontal plane. The holding arm 22 therefore extends laterally away from the main body 36 or the mounting unit 34. Furthermore, it can be seen that the majority of the holding arm 22 does not extend over the base surface 38. The holding arm 22 extends away from the base surface 38.

The pivot joints 44 and 46 are also driven by motor. The entire process of aligning the holding arm 22 in the space can therefore be driven by motor. The height of the holding arm 22 above the floor can therefore be adjusted by means of the column 47. The pivot joint 44 allows an angular position of the holding arm 22 in the horizontal plane to be set, and the pivot joint 46 allows the orientation of the robot arm interfaces 12, 16, 26 to be set.

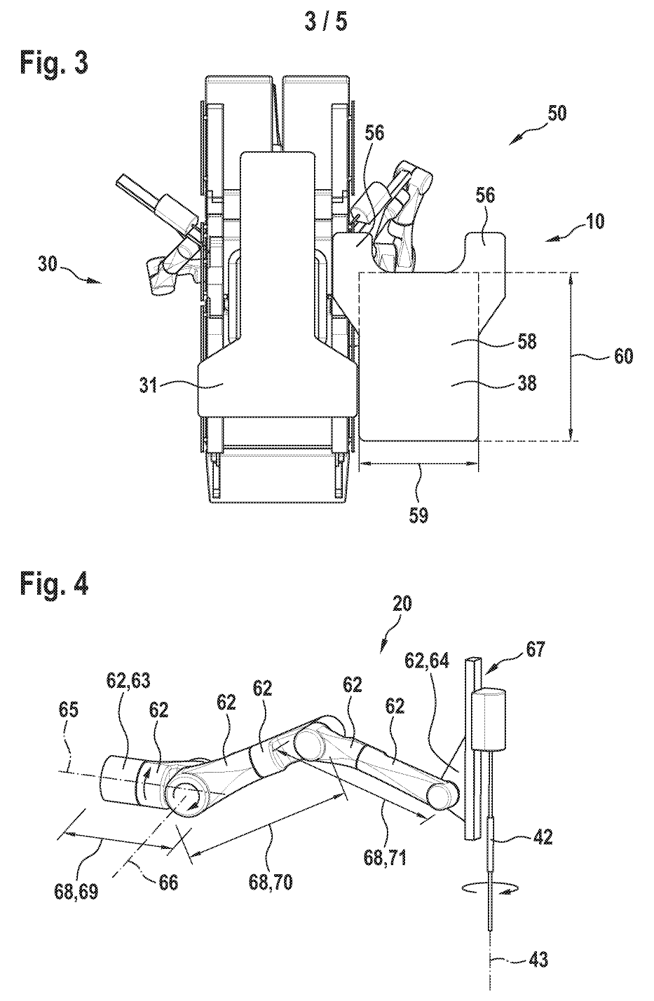

FIG. 3 is a schematic bottom view of the medical operating system 50 with the robot base 10 and of the operating table 30. The operating table 30 is placed on a floor (not shown) by means of a mounting surface 31. It can be seen that the base surface 38 of the robot base 10 is approximately the same size as the mounting surface 31. This compactness of the robot base 10 can be achieved by using the software RCM robot arms. These are characterized by their high compactness and low weight, especially compared to hardware RCM robot arms.

The base surface 38 has a rectangular bottom surface 58 with a width 59 of approximately 650 mm and a length 60 of approximately 800 mm. At two corners of a broad side of the bottom surface 58, two projections 56, which also belong to the base surface 38, extend laterally from the bottom surface 58 and along the longitudinal direction of the bottom surface 58. These extend in a preferred direction in which the robot arm interfaces (see above) of the holding arm are preferably oriented during use. This prevents the robot base 10 from tipping over, while simultaneously providing a space-saving design.

FIG. 4 is a schematic view of one of the software RCM robot arms 20. The robot arms 20 shown in FIG. 1 are identical. However, the medical instrument 42 fastened to each of the robot arms 20 may differ. Only a distal portion of the corresponding medical instrument 42 is inserted into the body cavity. The software RCM robot arm 20 is controlled in such a way that the medical instrument 42 is moved about the trocar point in the manner already described. The software RCM robot arm 20 has seven degrees of freedom. Six of these degrees of freedom are degrees of rotational freedom, and one is a degree of translational freedom. The medical instrument 42 can move linearly through the trocar point by means of the degree of translational freedom. By means of the degrees of rotational freedom, the medical instrument 42 can move about the trocar point without any lateral shifting taking place. The software RCM robot arm 20 is formed from a plurality of segments 62 - more precisely, seven segments 62 - which are mounted so as to be movable relative to one another. In particular, the segments 62 can be driven by motor to move relative to one another. The motors required therefor can be incorporated into the software RCM robot arm 20.

A proximal segment 62 is designed as a holding portion 63. The software RCM robot arm 20 can be coupled to a robot arm interface by means of the holding portion 63. The coupling is designed to be rigid. This means that no relative movement is provided between the holding portion 63 and the holding arm during a procedure.

A distal segment 64 is provided to support the medical instrument 42. This has a linear guide 67, which provides the degree of translational freedom. The medical instrument 42 is movable along the linear guide 67.

The seven segments 62 are rotatably coupled to one another. A rotational movement can be performed between each of the segments 62. The type of rotational movement, or the axis about which the rotational movement is performed, is designed to alternate from proximal to distal. This means that a rotational movement about a longitudinal axis 65 of a segment 62 is possible. The rotational movement, which is made possible by coupling each segment to a subsequent segment, is possible about an axis of rotation 66 that is perpendicular to the longitudinal axis 65. A rotational movement about a segment longitudinal axis is in turn possible for this segment and the following segment.

Two segments 62, excluding the distal segment 64, each form a robot arm portion 68. The holding portion 63 and the segment 62 directly coupled thereto form a first robot arm portion 69. This robot arm portion is approximately 250 mm long. The next two segments 62 form a second robot arm portion 70, which is approximately 380 mm long. The two segments 62 that then follow form a third robot arm portion 71, which is approximately 380 mm long. The distal segment 64 is approximately 470 mm long. It goes without saying that these dimensions may vary depending upon the robot arm and application and are to be understood only as examples.

The medical instrument 42 can in turn have one degree of rotational freedom and allow a rotational movement about its longitudinal axis 43. An end effector (not shown in detail) attached to the medical instrument 42 can thereby be aligned within the patient's body cavity. The medical instrument 42 may comprise an endoscope. The optics of the endoscope can be aligned accordingly.

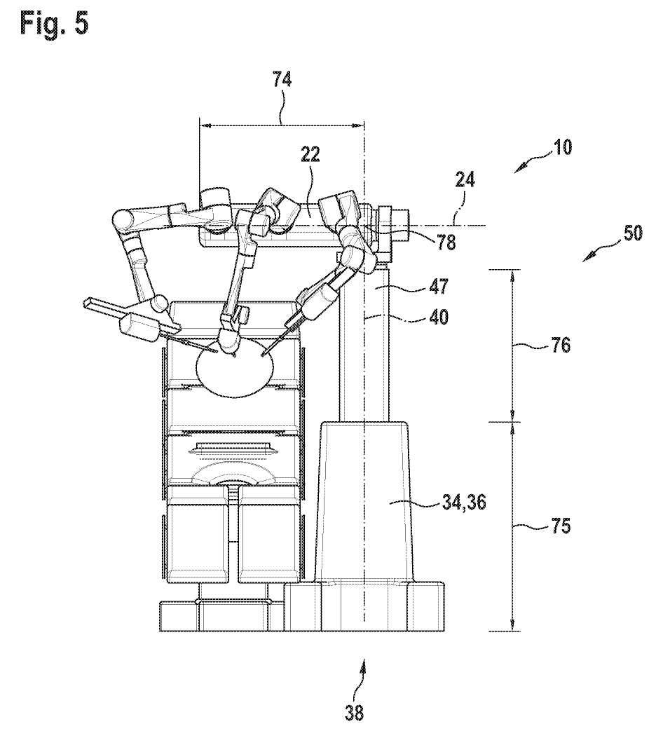

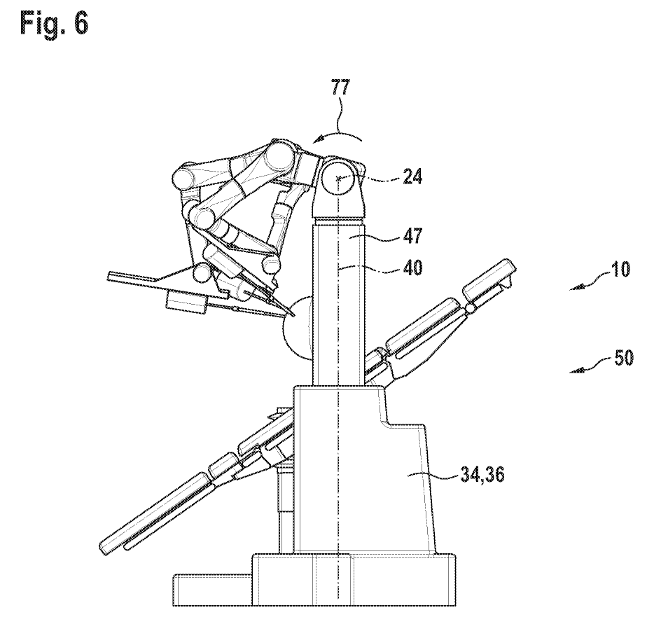

FIGS. 5 and 6 show further schematic views of the medical operating system 50 with the robot base 10. FIG. 5 shows a frontal view, and FIG. 6 a lateral view. It can be seen that the holding arm 22 extends with its longitudinal axis 24 perpendicular to the vertical axis 40. The holding arm 22 extends laterally beyond the base surface 38. The holding arm 22 shown has a length 74 of approximately 700 mm. Furthermore, the main body 36 or the mounting unit 34 of the robot base 10 can be seen. This has a height 75 of approximately 800 mm. The column 47 is shown with a length 76 of approximately 500 mm extending from the main body 36 or the mounting unit 34. The intersection 78 of the longitudinal axis 24 and the vertical axis 40 can be moved by the linear movement of the column 47 by a distance above the floor ranging from approximately 900 mm to 1,500 mm. As already described, the holding arm 22 can be rotated about the longitudinal axis 24 in the direction of the arrow 77 (see FIG. 6).

What is claimed is:

1. A robot base, comprising:
a first robot arm interface designed to couple a first robot arm , and
at least a second robot arm interface designed to couple a second robot arm,
characterized in that
the first robot arm interface and the second robot arm interface are each designed to couple a software RCM robot arm .

2. The robot base according to claim 1, characterized in that the position of the first robot arm interface relative to the second robot arm interface is fixed.

3. The robot base according to claim 1, characterized by a holding arm on which the first robot arm interface and the second robot arm interface are formed in a fixed position relative to one another.

4. The robot base according to claim 3, characterized in that the first robot arm interface and the second robot arm interface are arranged side-by-side along a longitudinal axis of the holding arm .

5. The robot base according to claim 3 , characterized by
at least a third robot arm interface which is designed to couple a third robot arm,
wherein the third robot arm interface is formed in a fixed position relative to the first and second robot arm interfaces on the holding arm .

6. The robot base according to claim 4, characterized in that the first robot arm interface , the second robot arm interface , and the third robot arm interface are arranged side-by-side on the holding arm so as to be at equal distances from one another along the longitudinal axis .

7. The robot base according to claim 3, characterized in that the holding arm can be arranged suspended above an operating table, in particular such that the robot arm interfaces can be arranged in an imaginary space extending vertically above the operating table and laterally delimited by the operating table .

8. The robot base according to claim 3, characterized by
a mounting unit for placement on a floor,
wherein the holding arm is connected to the mounting unit and has at least three, and in particular exactly three, degrees of freedom with respect to the mounting unit.

9. The robot base according to claim 8, characterized in that the holding arm can move linearly in a vertical direction relative to the mounting unit , in particular along a vertical axis which extends through the mounting unit .

10. The robot base according to claim 8 , characterized in that the holding arm is rotatable relative to the mounting unit about a vertical axis which extends in particular through the mounting unit .

11. The robot base according to claim 3, characterized in that the holding arm is rotatable about its longitudinal axis , wherein the longitudinal axis in particular is perpendicular to a vertical axis which extends through the mounting unit .

12. A medical operating system , comprising:
a robot base according to claim 1, wherein the first software RCM robot arm is coupled to the first robot arm interface.

13. The medical operating system according to claim 12, characterized in that the software RCM robot arm has at least seven degrees of freedom.

14. The medical operating system according to claim 13, characterized in that six of the degrees of freedom are degrees of rotational freedom.

15. The medical operating system according to claim 12, characterized by
a second software RCM robot arm which has a corresponding design to the first software RCM robot arm and is coupled to the second robot arm interface.
